(19) United States
(12) Patent Application Publication
(10) Pub. No.: US 2017/0014150 A1
(43) Pub. Date: Jan. 19, 2017

(54) INSTRUMENT FOR REMOVING BODY TISSUE FROM A FISTULOUS TRACT

(71) Applicant: A.M.I. Agency for Medical Innovations GmbH, Feldkirch (AT)

(72) Inventor: Martin Hohlrieder, Gotzis (AT)

(73) Assignee: A.M.I. Agency for Medical Innovations GmbH, Feldkirch (AT)

(21) Appl. No.: 15/208,797

(22) Filed: Jul. 13, 2016

(30) Foreign Application Priority Data
Jul. 13, 2015 (AT) A 460/2015

(51) Int. Cl. A61B 17/32

(52) U.S. Cl. CPC A61B 17/320016; A61B 2017/320052; A61B 2017/00991

(57) ABSTRACT

An instrument for removing body tissue from a fistulous tract (1) is provided, including a cutting tool (10) having a blade (12) and a guide rod (30) to be guided through the fistulous tract (1). The cutting tool (10) has a tubular embodiment and it is slideable onto the guide rod (30). The cutting tool (10) has a rear guiding portion (16) for displaceably guiding the cutting tool (10) on the guide rod (30) and a front cutting portion (11), at a front end of which the blade (12) is formed, and the internal diameter (15) thereof is greater than the external diameter (34) of the guide rod (30) in order to form a receptacle cavity (14) for receiving severed body tissue (5).

### INCORPORATION BY REFERENCE

The following documents are incorporated herein by reference as if fully set forth: Austrian Patent Application A460/2015, filed Jul. 13, 2015.

### BACKGROUND

The present invention relates to an instrument for removing body tissue from a fistulous tract, comprising a cutting tool having a blade.

Fistulae are not naturally pre-existing, tubular or tubular network-like connections between an inner hollow organ and other organs or the body surface of living beings. The body can form one or more fistulous tracts in order to remove the pus created in the case of an inflammation from an abscess.

Fistulae, for example in the region of the anus, so-called anal fistulae, are very uncomfortable for the affected party. In this case, the fistulous tract connects the rectum with the body surface in the region of the anus of the patient. Anal fistulae often have an epithelial lining which forms a fistulous wall of the fistulous tract and which has a thickness of approximately 0.5 mm or more. The epithelial lining hinders the independent growing together and healing of the fistula. The length of the fistulous tract of anal fistulae often lies in the range from 2 cm to 6 cm. The diameter of a fistulous tract is generally more than 2 mm.

Various therapies for treating fistulae, in particular anal fistulae, are known. Here, the pus is initially drained from the fistula by means of a thread drainage where necessary. To this end, a thread is introduced into the fistulous tract with the aid of a rod-shaped insertion aid and both ends of the thread are tied into a knot outside of the body of the patient. Where possible, fistulae are then cut out or split while sparing the sphincter muscle in order to enable healing of the fistula. This is problematic, particularly in the case of fistulous tracts near the sphincter muscle, since a radical removal can put the continence of the patient at risk.

The extent of body tissue to be removed by operation can be reduced by inserting a fistula closure, e.g. made of a resorbable implant, into the fistulous tract from the inside. A further fistula closure technique consists of the thermal treatment of the fistulous tract, e.g. by means of a laser, in order to destroy and occlude the fistulous tract.

A further therapy form consists of manually ablating the fistulous wall by means of a curette, i.e. a rod-shaped instrument with a widened front end, on which a blade is formed, in order to enable subsequent healing of the fistula. To this end, it is necessary to cut out the external fistula portions over a large area in the region of the opening, for example with a funnel shape, in order to enable healing of the fistula from the inside. A multiplicity of curettes are required for ablating the fistulous wall as these quickly become blunt. The intervention is time-intensive and requires great concentration of the staff implementing this so that the fistulous wall is completely ablated. If residues of the fistulous wall remain in the fistulous tract, new inflammations may arise, as a result of which the fistula often cannot heal in a satisfactory manner.

### SUMMARY

It is an object of the invention to provide an advantageous instrument of the type set forth at the outset, which enables a reliable and simple removal of body tissue from a fistulous tract.

This is achieved by an instrument having one or more features of the invention.

The instrument according to the invention has a guide rod to be guided through the fistulous tract. Furthermore, a cutting tool with a tubular embodiment is slideable onto the guide rod. The cutting tool has a rear guiding portion for displaceably guiding the cutting tool on the guide rod and a front cutting portion. The blade is formed at a front end of the cutting tool. The internal diameter of the cutting portion is greater than the external diameter of the guide rod in order to form a receptacle cavity for receiving severed body tissue.

Firstly, the guide rod serves for the so-called preparation of the fistulous tract, i.e. for ensuring that the fistulous tract is appropriately aligned prior to carrying out the cutting process. Here, the external diameter of the guide rod is preferably selected in such a way that the fistulous tract is spanned on the guide rod, i.e. that the fistulous tract is slightly widened by the inserted guide rod, wherein the fistulous wall expediently rests against the guide rod over the whole longitudinal extent of the fistulous tract. Secondly, the guide rod also serves to guide the cutting tool along the longitudinal extent of the guide rod. Within the scope of the invention, the guide rod can also be referred to as rod or guide bar. Preferably, the guide rod has a straight embodiment.

The risk of unwanted injury to the surrounding body tissue can be reduced by the defined guidance of the cutting tool on the guide rod in the direction of the longitudinal center axis of the guide rod since the degree of freedom of the cutting tool in relation to the guide rod is advantageously restricted to one axial direction (in the direction of the longitudinal center axis of the guide rod) and to a rotation about the longitudinal center axis of the guide rod. Moreover, as a result thereof, body tissue can be removed in a defined manner.

Preferably, in an axial view of the cutting tool, the cutting edge of the blade of the cutting tool lies on a circle which lies concentrically with the longitudinal center axis of the guide rod when the cutting tool is pushed onto the guide rod. By twisting the cutting tool about the longitudinal center axis of the guide rod and by simultaneously advancing it in the direction of the longitudinal center axis, the fistulous wall surrounding the fistulous tract can be severed from the surrounding body tissue as a geometrically defined hollow cylinder.

The blade can have a continuous circular embodiment, i.e. the cutting edge of the blade is circular in that case. However, by way of example, it would also be conceivable and feasible for the cutting edge to have a serrated edge. Even in the case of a serrated edge, the cutting edge of the blade preferably lies on a circle as seen in an axial view of the cutting tool.

The cutting tool with the tubular embodiment could also be referred to as a cutting tube. Cutting tubes for use in medical applications are known per se, for example for laparoscopic hysterectomy and for myoma enucleation. So-called morcellators are known in the field of minimally invasive surgery; these serve to comminute large tissue parts or organs to be removed from the body interior. Here, a motor-driven cutting tube of the morecellator, which is surrounded by a protective tube, rotates with a high rotational speed. The comminuted tissue parts are transported to the outside through the interior cavity of the cutting tube.

In relation to the direction of the longitudinal center axis of the cutting tool, the guiding portion preferably has a length of at least 1 cm, preferably at least 3 cm, by means of which the sliding guidance of the cutting tool on the guide rod is carried out. By way of example, the length can be in the region of 5 cm. As a result, stable guidance of the cutting tool can be achieved.

Preferably, the instrument has a counter-holding piece, which is connectable to a front end of the guide rod and which is removable therefrom, wherein the counter-holding piece has a stop for the blade restricting the displacement travel of the cutting tool on the guide rod. After guiding the guide rod through the fistulous tract, the counter-holding piece can be connected to the guide rod by medical staff by means of medical instruments. By way of example, latching connection or other connection elements known from the prior art are suitable for the connection. A screw-in connection is particularly preferable.

The body tissue to be removed is supported by the counter-holding piece on the side lying opposite to the blade, wherein the restricting stop enables a particularly clean final cut for the blade, i.e. complete separation of the fistulous wall or of the body tissue to be removed. Preferably, the diameter of the restricting stop is greater than the circle diameter of the circle on which the cutting edge of the blade lies in order to prevent inadvertent pulling of the counter-holding piece into the fistulous tract.

Advantageously, the instrument for inserting the guide rod into the fistulous tract has a rod-shaped and bendable insertion aid, which is connectable to a front end of the guide rod and detachable therefrom. The insertion aid could also be referred to as insertion probe and it is expediently plastically deformable by hand in a simple manner. As a result of this, it is possible to probe curved fistulous tracts in particular, as a result of which the guide rod can be inserted into such a curved fistulous tract under straightening of the fistulous tract. The diameter of the insertion aid is expediently less than the diameter of the fistulous tract.

Thus, the guide rod expediently has a greater flexural rigidity than the insertion aid, and so the insertion aid is more easily bendable, in particular much more easily bendable, than the guide rod. Preferably, the guide rod is not deformed when the instrument is used. This renders it possible to maintain the coaxiality of the blade of the cutting tool in respect of the guide rod during the cutting process. The external diameter of the cutting rod is expediently more than 3 mm. As a result of this, it is possible to achieve sufficient stability against bending of the guide rod for the purposes of the concentric guide of the blade.

It is advantageous if the guide rod has a handle to make it easier for the user to hold the guide rod, said handle, as seen in a cross section orthogonal to the longitudinal center axis of the guide rod, having an external contour deviating from the circular form. As a result of this, it is possible, for example, to simplify the twisting of the guide rod, for example for screwing-on the counter-holding piece or the insertion aid.

The guide rod preferably has a length of at least 6 cm, particularly preferably of at least 15 cm.

Expediently, the cutting tool has an operating handle to make it easier to operate the cutting tool, said operating handle, as seen in a cross section orthogonal to the longitudinal center axis of the cutting tool, having an external contour deviating from the circular form. By way of the operating handle, it is possible to twist the cutting tool relative to the guide rod in order to carry out the cutting movement. The movement of the cutting tool in the direction of the longitudinal center axis of the guide rod can also be simplified thereby.

Advantageously, the length, as measured in the direction of the longitudinal center axis of the cutting tool, of the cutting section of the cutting tool, and hence the length of the receptacle cavity formed when the cutting tool is pushed onto the guide rod, is at least 2 cm, preferably at least 4 cm, particularly preferably at least 6 cm. The length of the cutting portion, and hence the length of the receptacle cavity formed in the state of the cutting tool pushed onto the guide rod, advantageously at least corresponds to the length of the fistulous wall of the fistulous tract to be removed.

Preferably, the difference between the circle diameter of the circle on which the cutting edge of the blade lies in an axial view onto the cutting tool and the external diameter of the guide rod is at least 1 mm, particularly preferably at least 2 mm. As a result, a sleeve-shaped tissue region with a corresponding thickness can be ablated in the case of a fistulous tract lying against the guide rod.

### DETAILED DESCRIPTION OF THE PREFERRED EMBODIMENTS

Further features and details of the invention are explained on the basis of the exemplary embodiment, shown in the figures, of an instrument for removing body tissue from a fistulous tract and on the basis of exemplary schematic illustrations of the severing of body tissue in the region of an anal fistula. In detail:

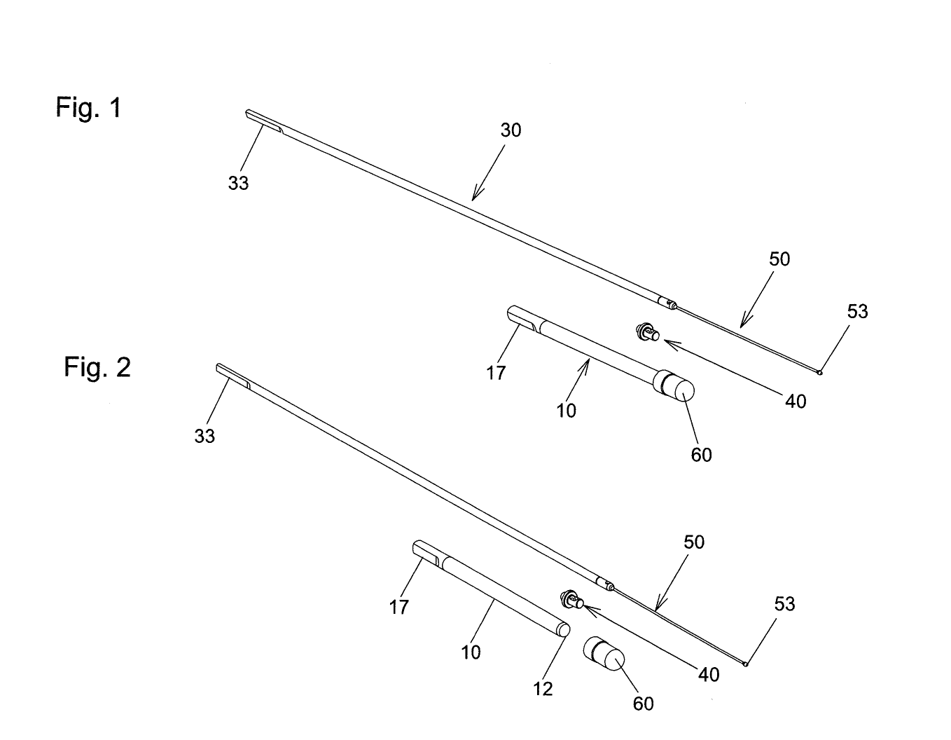

FIG. 1 shows an isometric view of an instrument according to the invention in the supplied state;

FIG. 2 shows the instrument in accordance with FIG. 1 with a protective cap removed from the cutting tool;

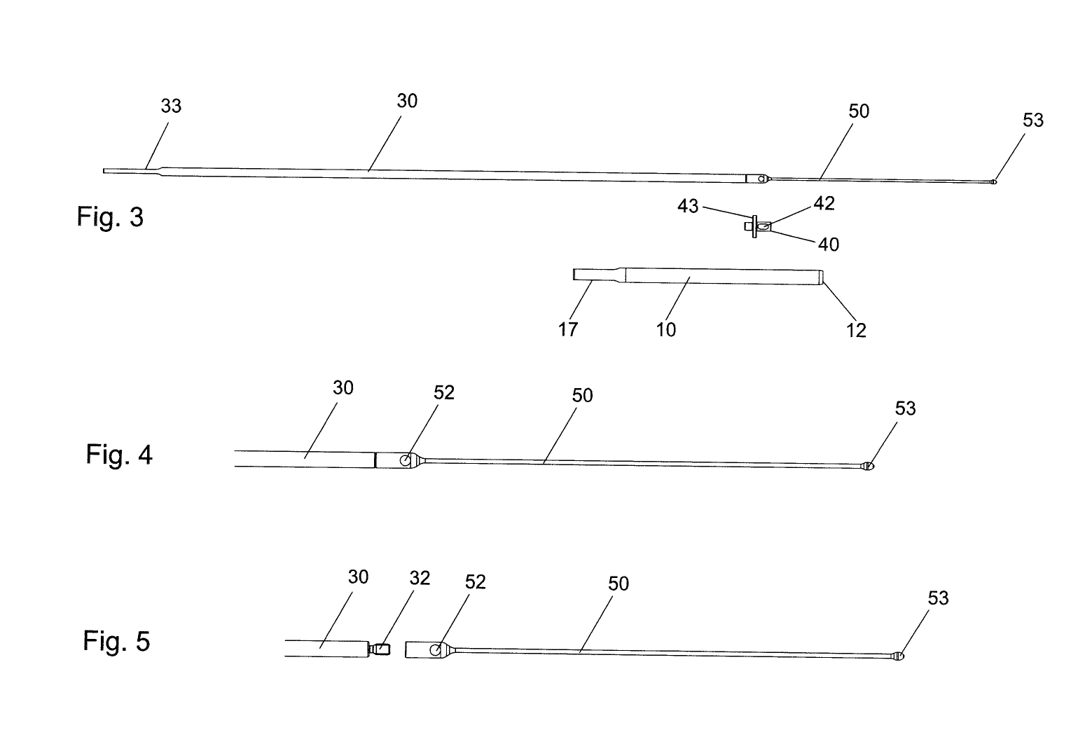

FIG. 3 shows a side view of the instrument in accordance with FIG. 2 without a protective cap;

FIG. 4 shows a detailed view of the insertion aid in accordance with FIG. 3 connected to the guide rod;

FIG. 5 shows the insertion aid removed from the guide rod;

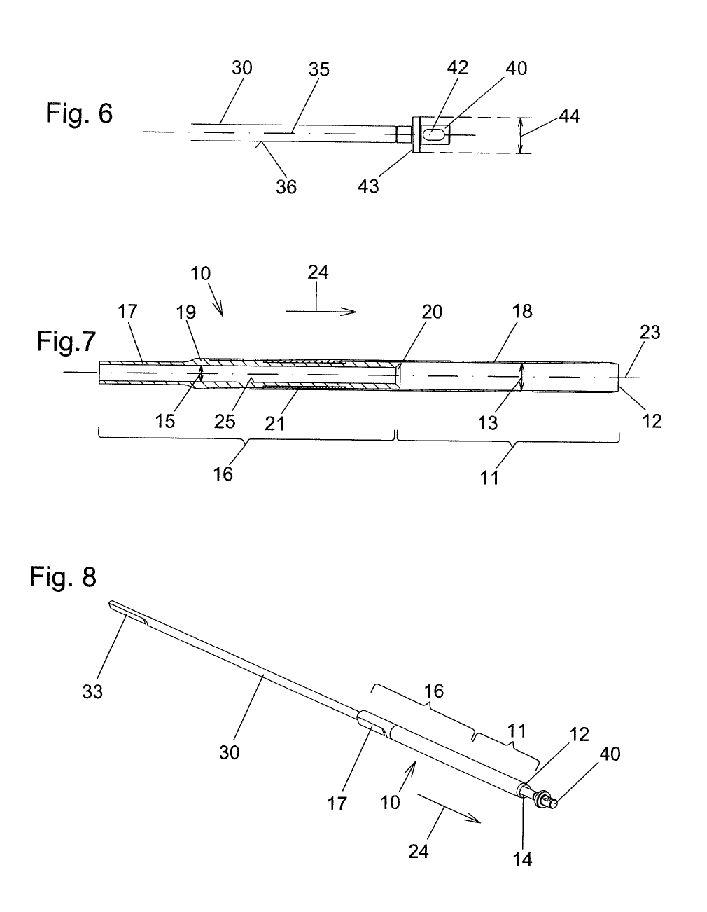

FIG. 6 shows a detailed view of the counter-holding piece screwed onto the guide rod;

FIG. 7 shows a longitudinal center section of the cutting tool (sectional plane parallel to the plane of the drawing in FIG. 3);

FIG. 8 shows an isometric view of the cutting tool pushed onto the guide rod, including the counter-holding piece;

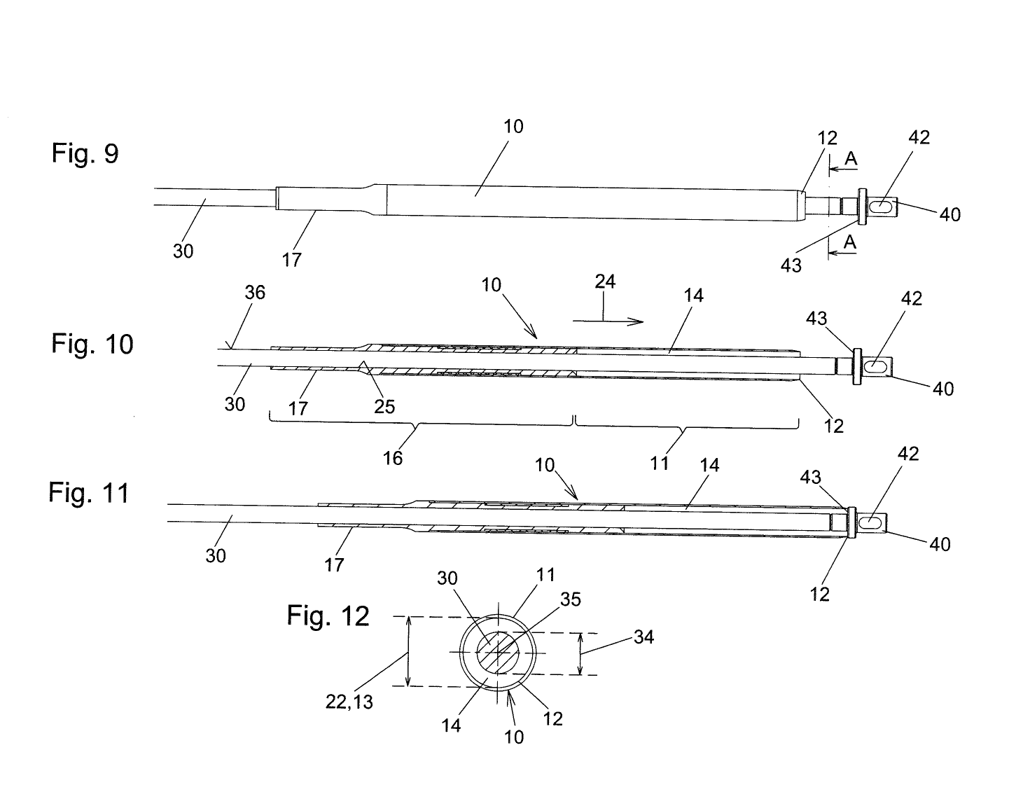

FIG. 9 shows a side view of a front portion of the instrument in accordance with FIG. 8;

FIG. 10 shows a longitudinal center section through the region of the instrument depicted in FIG. 9;

FIG. 11 shows the longitudinal center section analogous to FIG. 10 with a cutting tool advanced up to the stop;

FIG. 12 shows the section A-A in accordance with FIG. 9;

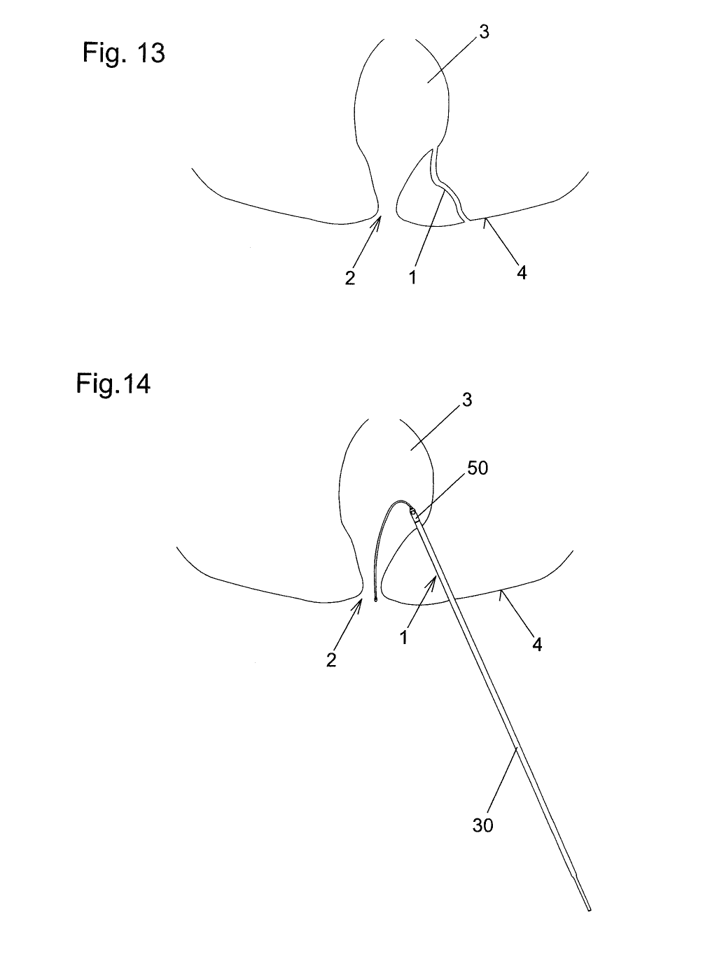

FIG. 13 shows a schematic sectional illustration of a fistula in the region of the anus;

FIGS. 14 to 16 show schematic illustrations of various steps for severing body tissue in the region of the fistulous tract in accordance with FIG. 13; and

FIG. 17 shows the detail B in accordance with FIG. 16 in a magnified illustration.

### DETAILED DESCRIPTION OF THE PREFERRED EMBODIMENT

The instrument for removing body tissue from a fistulous tract 1 has a tubular cutting tool 10. The form of the cutting tool 10 could also be referred to as sleeve-shaped. In the exemplary embodiment, the cutting tool 10 is formed by a first tube piece 18 and a second tube piece 19, which are rigidly connected to one another, preferably in a cohesive manner, particularly preferably by means of an adhesive 21, for example by means of a two-component adhesive known per se. By way of example, the connection could also be carried out by pressing. Hence, in the exemplary embodiment, the cutting tool 10 substantially is formed of metal (i.e. apart from the adhesive 21). However, this is only one possible embodiment of a cutting tool 10 according to the invention. By way of example, it could also be manufactured integrally from a single blank, in particularly from metal. In other embodiments, the second tube piece 19 could, for example, also be manufactured from plastic and the first tube piece 18 could be manufactured from metal or ceramics.

In respect of a cutting direction 24 of the cutting tool 10, the cutting tool 10 has a front cutting portion 11, on which a blade 12 is formed at a front end by virtue of the wall of the cutting tool 10 tapering at the front end of the cutting portion 11 to form a cutting edge. The cutting edge lies on a circle, cf. FIG. 12, in an axial view of the cutting tool 10, i.e. as seen orthogonally to the longitudinal center axis 23 of the cutting tool 10. In the exemplary embodiment, the cutting edge of the blade 12 lies in a plane over the entire circumference thereof; however, it could, for example, also have a serrated edge.

The circle diameter 22 of the circle on which the cutting edge of the blade 12 lies is less than 1 cm; the circle diameter 22 is 0.6 cm in the exemplary embodiment. Depending on the cross section of the fistulous tract 1 or the thickness of the fistulous wall, the circle diameter 22 can be selected by selecting an appropriate cutting tool 10.

In the exemplary embodiment, the blade 12 has a conic-frustum-shell-shaped external side—cf. FIGS. 7 and 8—but it could also have an embodiment with, for example, a bevel on the inside or on both sides. In the supplied state, the protective cap 60 of the instrument protects the blade 12 from dirtying or damage; cf. FIG. 1.

In relation to the cutting direction 24, the cutting tool 10 has a rear guiding portion 16 for displaceably guiding the cutting tool 10 onto the guide rod 30 yet to be explained in more detail below. The internal diameter 15 of the guiding portion 16 over the whole axial extent of the guiding portion 16 is less than the internal diameter 13 of the cutting portion 11 over the whole axial extent of the cutting portion 11; cf. FIG. 7. Expediently, a countersink (=chamfer) 20 is provided in a transition region between the cutting portion 11 and the guiding portion 16; this simplifies pushing the cutting tool 10 onto the guide rod 30. Such a countersink can also be dispensed with.

In order to simplify the handling of the cutting tool 10 for the operator, the cutting tool 10 expediently has at the external side of the guiding portion 16 an operating handle 17 for simplifying the operation of the cutting tool 10. As seen in a cross section orthogonal to the longitudinal center axis 23 of the cutting tool 10, the operating handle 17 has an external contour deviating from the circular form. In the exemplary embodiment, the operating handle 17 is formed by two opposite flat spots; cf. FIG. 3.

The guide rod 30 is elongate and straight. Expediently, it has a circular cross section over the majority of the longitudinal extent thereof; cf. FIG. 3 and FIG. 12. In the exemplary embodiment, the length of the guide rod 30 is 25 cm.

The cutting tool 10 can be pushed onto the guide rod 30, with the guiding portion 16 of the cutting tool 10 being displaceably guided with the inner surface 25 thereof on the external surface 36 of the guide rod 30. Thus, the inner surface 25 of the rear guiding portion 16 interacts with the external surface 36 of the guide rod 30 to form a sliding guide of the cutting tool 10 on the guide rod 30. The inner surface 25 of the guiding portion 16 preferably has a cylinder-shell-shaped embodiment.

In the state where the cutting tool 10 is pushed onto the guide rod 30, the cutting tool 10 and the guide rod 30 are coaxial, i.e. the longitudinal center axis 23 of the cutting tool 10 and the longitudinal center axis 35 of the guide rod 30 lie on a common straight line. In the state pushed onto the guide rod 30, the circle on which the cutting edge of the blade 12 lies also lies coaxially with the guide rod 30; cf. FIG. 12.

In the exemplary embodiment, the guide rod 30 is formed of metal. It remains virtually non-deformed during use. As a result, the coaxial property of the blade 12 in relation to the guide rod 30 can be ensured during the cutting process. Using this, it is possible to achieve uniform severing of the fistulous wall over the whole longitudinal extent of the fistulous tract 1, as explained in more detail below.

Together, the internal diameter 15 of the cutting portion 16 and the external diameter 34 of the guide rod 30 form a loose fit in order to form a sliding guide of the cutting tool 10 on the guide rod 30. The difference between the internal diameter 15 of the guiding portion 16 of the cutting tool 10 and of the external diameter 34 of the guide rod 30 is expediently less than 0.2 mm (at least over the majority of the longitudinal extent of the guide rod 30, over which the latter expediently has a circular cylindrical embodiment).

A handle 33 to make it easier for the surgeon to hold the guide rod 30 is preferably embodied at the rear end of the guide rod 30. As seen in a cross section orthogonal to the longitudinal center axis 35 of the guide rod 30, the guide rod 30 has an external contour deviating from the circular form in the region of the handle 33. In the exemplary embodiment, the handle 33 is formed by two opposite flat spots on the guide rod 30; cf. FIG. 3.

Both the handle 33 of the guide rod 30 and the operating handle 17 of the cutting tool 10 could have an external contour with a different embodiment than the one formed by the flat spots of the exemplary embodiment, e.g. a star-shaped external contour, in other embodiments according to the invention.

The internal diameter 13 of the cutting portion 11 of the cutting tool 10 is greater than the external diameter 34 of the guide rod 30 over the entire axial extent of the cutting tool 11. A receptacle cavity 14 for receiving severed body tissue 5 is thus formed when the cutting tool 10 is pushed onto the guide rod 30. This receptacle cavity 14 is therefore restricted to the inside by the external surface 36 of the guide rod 30 and to the outside by the inner surface 25 of the cutting portion 11 of the cutting tool 10. The receptacle cavity 14 is therefore sleeve-shaped or tubular and open to the front (in relation to the cutting direction 24); cf. FIGS. 10 and 12. The receptacle cavity 14 directly adjoins the cutting edge of the blade 12. The circle diameter 22 of the circle on which the cutting edge of the blade 12 lies corresponds to the internal diameter 13 of the cutting portion 11 of the cutting tool 10 in the exemplary embodiment.

The difference between the internal diameter 13 of the cutting potion 11 and the external diameter 34 of the guide rod 30 is preferably at least 1 mm, particularly preferably at least 2 mm.

The external diameter of the cutting tool 10 is constant over the cutting portion 11 and it is at least not greater than in the cutting portion 11 over the guiding portion 16.

Moreover, the instrument has a rod-shaped and bendable insertion aid 50, which is connectable to, and removable from, a front end of the guide rod 30. For the purposes of connection with the guide rod 30, the insertion aid 50 has a threaded bore in the exemplary embodiment (not visible in the figures). A corresponding threaded pin 32 is embodied at the front end of the guide rod 30; cf. FIG. 5. At the front end of the insertion aid 50, the latter has a rounded-off head 53, which eases the insertion of the insertion aid 50 into the fistulous tract 1. During the insertion into the fistulous tract 1, the insertion aid 50, as a result of the resilience thereof, can follow the course of the fistulous tract 1 or there can also already be a certain amount of straightening of the fistulous tract 1 when the insertion aid 50 is inserted into the fistulous tract 1.

In order to simplify the screwing of the insertion aid 50 onto, or from, the front end of the guide rod 30, the insertion aid 50 can have a recess 52 which simplifies the manipulation by means of a medical instrument. In the supplied state, the insertion aid 50 is expediently already connected to the guide rod 30 in order to avoid an additional work step of screwing on the insertion aid 50; cf. FIG. 1.

Furthermore, the instrument has a counter-holding piece 40, which is connectable to, and removable from, the guide rod 30; cf. FIG. 6. Analogously to the insertion aid 50, the counter-holding piece 40 has a threaded bore (not visible in the figures), which is connectable to, and again removable from, the threaded pin 32 at the front end of the guide rod 30 by screwing.

The counter-holding piece 40 has a stop 43 which restricts the displacement travel of the cutting tool 10 on the guide rod 30 in the cutting direction 24. In the state where the cutting tool 10 is pushed to the furthest possible extent in the cutting direction 24 on the guide rod 30, the blade 12 abuts on the stop 43; cf. FIG. 11.

The counter-holding piece 40 can have a recess 42 in order to simplify the screwing-on of the counter-holding piece 40 in the interior of the body during the intervention. By way of example, the surgeon, e.g. by means of forceps, can hold the counter-holding piece 40 and rotate the guide rod 30 in order thus to establish the connection with the guide rod 30.

Preferably, the cutting edge of the blade 12 circumferentially abuts on the stop 43 when displacing the cutting tool 10 in order to enable clean severing at the end of the displacement travel of the cutting tool 10 on the guide rod 30; cf. FIG. 11. In the exemplary embodiment, the diameter 44 is only slightly larger than the circle diameter 22; however, it could also be significantly larger in other embodiments of the invention.

Below, the process of removing body tissue from a fistulous tract 1 in the region of the anus is explained on the basis of FIGS. 13 to 17. The fistulous tract 1 forms a channel between the rectum 3 and the body surface 4. The course of the fistulous tract 1 is not straight, as generally found in practice; cf. FIG. 13. In the depicted example, the length of the fistulous tract is approximately 5 cm. The fistulous wall limiting the fistulous tract 1, which is not drawn separately in the drawings, is an epithelial lining, i.e. it is formed by a type of thin skin, the thickness of which usually lies in the range of 0.5 mm to 1 mm.

Initially, the guide rod 30 is inserted into the fistulous tract 1 by means of the insertion aid 50. The fistulous tract 1 is straightened by the inserted guide rod 30; cf. FIG. 14. It should be noted here that the external diameter 34 of the guide rod 30 is expediently selected to be greater than the diameter of the fistulous tract 1. As a result, the fistulous wall of the fistulous tract 1, in the entirety thereof, abuts completely against the guide rod 30 in the inserted state of the guide rod 30.

In a next step, the insertion aid 50 is removed from the guide rod 30. To this end, the insertion aid 50 is held in a manner secured against rotation by way of a medical instrument, e.g. by way of forceps, while the guide rod 30 is rotated in relation to the fistulous tract 1 and the insertion aid 50, for example by means of the handle 33. After screwing-off and removing the insertion aid 50, the counter-holding piece 40 is connected to the front end of the guide rod 30 situated in the interior of the body. To this end, the guide rod 30 can also be expediently rotated by means of the handle 33 in relation to the secured counter-holding piece 40. Expediently, the counter-holding piece 40 also forms a type of anchor in the state where it is connected to the guide rod 30 in order to prevent the front end of the guide rod 30 from slipping into the fistulous tract 1 during the cutting process.

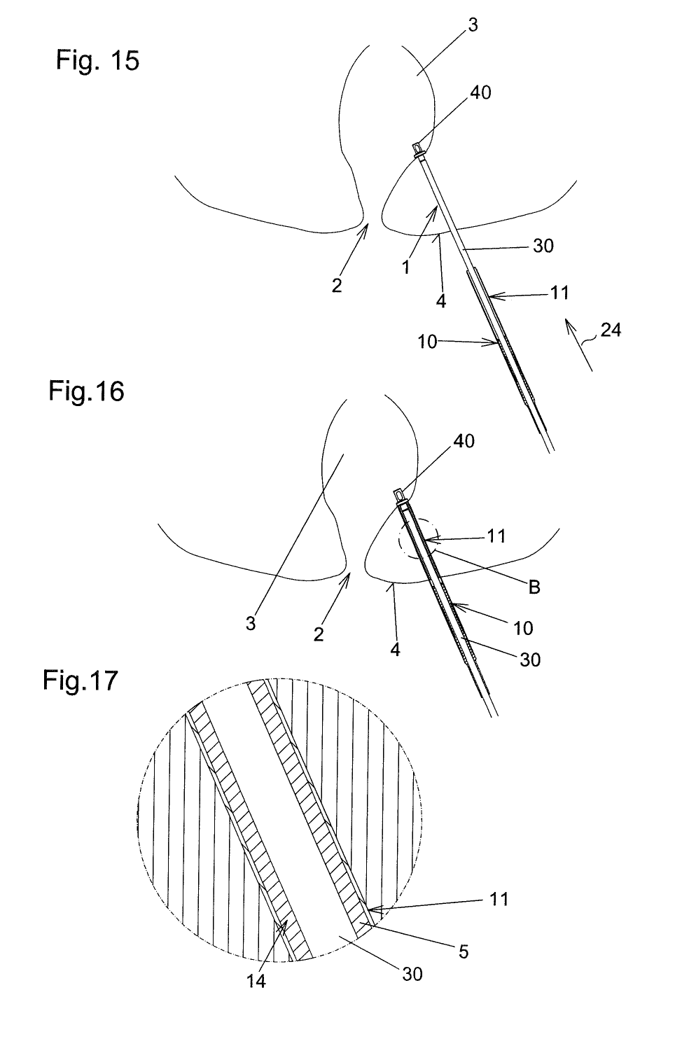

In the subsequent work step, the cutting tool 10 is pushed onto the guide rod 30; cf. FIG. 15. During the subsequent cutting process, the cutting tool 10 is expediently rotated in relation to the guide rod 30 in addition to the movement in the cutting direction 24 in order to enable a clean cut. The body tissue 5 already severed during the cutting process is successively received in the receptacle cavity 14 between the cutting portion 11 of the cutting tool 10 and the guide rod 30; cf. FIGS. 16 and 17.

At the end of the displacement travel of the cutting tool 10, the blade 12 of the cutting portion 11 abuts the stop 43 of the counter-holding piece 40. Situated in the receptacle cavity 14 is the body tissue 5 which was completely severed from the surrounding tissue of the body and which comprises the epithelial lining, i.e. the fistulous wall of the fistulous tract 1.

In a last work step not shown separately here, the guide rod 30 with the cutting tool 10 and the severed body tissue 5 is guided out of the body. The counter-holding piece 40 can be removed in advance, particularly if use is made of a counter-holding piece 40, the diameter 44 of which is significantly greater than the external diameter of the cutting tool 10. Alternatively, particularly if the diameter of the counter-holding piece 40 approximately corresponds to the circle diameter 22, the counter-holding piece 40 could be pulled out of the body of the patient together with the guide rod 30 and the cutting tool 10.

After removing the fistulous wall, fresh, perfused body tissue is present in the region of the fistulous tract 1 which has now been widened in terms of the cross section thereof. The perfused tissue enables growing together and healing of the fistula after the intervention.

In the shown exemplary embodiment, the difference between the circle diameter 22 of the circle on which the cutting edge of the blade 12 lies and the external diameter 34 of the guide rod 30 is 2 mm. As a result, it is possible to remove the fistulous wall (which has e.g. a thickness of 0.5 mm) and body tissue adjoining the fistulous wall (e.g. with a thickness of 0.5 mm) in the entirety thereof over the whole longitudinal extent of the fistulous tract 1. Expressed differently, sleeve-shaped body tissue, with a wall thickness of 1 mm in the exemplary embodiment, can be removed by means of the illustrated instrument.

Instruments with appropriate dimensions can be provided depending on the length and diameter of the fistulous tract to be treated and depending on the thickness of the fistulous wall.

### LIST OF REFERENCE SIGNS

1 Fistulous tract
2 Anus
3 Rectum
4 Body surface
5 Severed body tissue
10 Cutting tool
11 Cutting portion
12 Blade
13 Internal diameter
14 Receptacle cavity
15 Internal diameter
16 Guiding portion
17 Operating handle
18 First tube piece
19 Second tube piece
20 Countersink
21 Adhesive
22 Circle diameter
23 Longitudinal center axis
24 Cutting direction
25 Inner surface
30 Guide rod
31 Longitudinal center axis
32 Threaded pin
33 Handle
34 External diameter
35 Longitudinal center axis
36 External surface
40 Counter-holding piece
42 Recess
43 Stop
44 Diameter
50 Insertion aid
52 Recess
53 Head
60 Protective cap

1. An instrument for removing body tissue from a fistulous tract, comprising:
a cutting tool having a blade, the cutting tool being embodied tubular;
a guide rod adapted to be guided through the fistulous tract,
the cutting tool is slideable onto the guide rod and has a rear guiding portion for displaceably guiding the cutting tool on the guide rod and a front cutting portion, the blade being formed at a front end of the cutting tool, and an internal diameter of the blade is greater than an external diameter of the guide rod in order to form a receptacle cavity adapted to receive severed body tissue.

2. The instrument according to claim 1, wherein, in an axial view of the cutting tool, the cutting edge of the blade lies on a circle which lies concentrically with the guide rod when the cutting tool is pushed onto the guide rod.

3. The instrument according to claim 2, wherein the circle has a circle diameter of at most 1 cm.

4. The instrument according to claim 1, further comprising a counter-holding piece, which is connectable to a front end of the guide rod and which is removable therefrom, the counter-holding piece has a stop for the blade that restricts a displacement travel of the cutting tool on the guide rod.

5. The instrument according to claim 1, wherein, in relation to a direction of a longitudinal center axis of the cutting tool, the guiding portion has a length of at least 1 cm.

6. The instrument according to claim 1, further comprising a rod-shaped and bendable insertion aid that is connectable to a front end of the guide rod and detachable therefrom.

7. The instrument according to claim 1, wherein the guide rod has a handle adapted for a user to hold the guide rod, said handle, as seen in a cross section orthogonal to a longitudinal center axis of the guide rod, having an external contour deviating from a circular form.

8. The instrument according to claim 1, wherein the receptacle cavity formed in a state where the cutting tool is pushed onto the guide rod has a length of at least 2 cm in relation to a direction of a longitudinal center axis of the cutting tool.

9. The instrument according to claim 1, wherein the guide rod has a length of at least 6 cm.

10. The instrument according to claim 1, wherein a difference between an internal diameter of the cutting potion and an external diameter of the guide rod is at least 1 mm.

11. The instrument according to claim 1, wherein the cutting tool has an operating handle for operating the cutting tool, said operating handle, as seen in a cross section orthogonal to the longitudinal center axis of the cutting tool, having an external contour deviating from a circular form.
